(19) United States
(12) Patent Application Publication
(10) Pub. No.: US 2023/0349084 A1
(43) Pub. Date: Nov. 2, 2023

(54) Nonwoven Web Containing a High Strength Spunblown Layer

(71) Applicant: Kimberly-Clark Worldwide, Inc., Neenah, WI (US)

(72) Inventors: Bryan D. Haynes, Jasper, GA (US); Allen F. Vater, Alpharetta, GA (US); Simon K. Poruthoor, Alpharetta, GA (US); Karen Goeders, Alpharetta, GA (US); Scott R. Lange, Appleton, WI (US)

(21) Appl. No.: 17/911,487

(22) PCT Filed: Apr. 23, 2020
(86) PCT No.: PCT/US2020/029459

§ 371 Date: Sep. 14, 2022

(51) Int. Cl. D04H 5/08; D04H 3/011; D04H 3/16; D04H 3/03

(52) U.S. Cl. CPC D04H 5/08; D04H 3/011; D04H 3/16; D04H 3/03; D10B 2401/063; D10B 2509/02; A61F 2013/15967

(57) ABSTRACT

The present invention discloses a nonwoven web and methods for manufacturing the nonwoven web. The present invention specifically discloses a nonwoven web that includes a co-forming process wherein a first gas stream includes meltblown fibers that are extruded from a first die head, a second gas stream that includes spunblown fibers that are extruded from a second die head and a third die head that includes pulp fibers that are extruded from a pulp nozzle. By sandwiching spunblown fibers between meltblown fibers during the co-forming processes, a nonwoven web having improved cross-machine direction tensile strength is formed.

### BACKGROUND

Nonwoven webs can be formed through different processes. One such process is generally referred to as a co-forming process. Co-forming involves, for example, melt spinning continuous fibers from polymer resin, and simultaneously, directing an air stream with entrained distributed cellulose fibers, typically into a co-forming box or similar blending apparatus, blending the fibers, and directing the blend along to a collecting/forming structure such as a moving forming belt. The fibers may be spun and introduced into the airstream carrying cellulose fibers, or an airstream carrying the cellulose fibers may be introduced into the pathway of the fibers following spinning, the effect being the deposit of an entangled blend of fibers onto the moving forming structure and accumulation thereof forming a cohesive nonwoven web. The fibers in the coformed nonwoven web provide a matrix that holds the fibers in place within the web and thereby help maintain structural integrity.

The nonwoven web should be strong enough to maintain structural integrity in use, but also soft enough to give a pleasing and comfortable tactile sensation to the user. In addition, the nonwoven web should have suitable strength, absorbency, porosity and surface texture to be effective in absorbing and retaining bodily exudates. Nonwoven web strength is measured by tensile strength. Additionally, cost and environmental sustainability concerns impose pressure to further improve nonwoven webs to enable improved tensile strength and less material to be used without compromising other important properties such as absorbency and surface texture.

The types of fibers used and the placement of fibers in the co-forming process are important factors for achieving desired tensile strength in the finished product.

As such, a need exists for a nonwoven web having increased strength without increasing the basis weight of the nonwoven web for use in a variety of applications. Accordingly, it is an object of the present invention to provide a nonwoven web having increased tensile strength.

### SUMMARY

Generally, the present invention relates to making a nonwoven web having increased tensile strength in a cross-direction by introducing reinforcing fibers into a fiber matrix of the nonwoven web through a co-forming process. More specifically, the present invention relates to a method for manufacturing a nonwoven web that includes providing a forming surface that is traveling in a machine-direction (MD). The present disclosure incorporates a co-forming process that includes a first and second meltblown die head disposed above the forming surface. The first meltblown die head extrudes a first gas stream and a second meltblown die head extrudes a second gas stream. The first gas stream includes meltblown fibers and the second gas stream includes spunblown fibers. Further, a pulp nozzle is disposed above and perpendicular to the forming surface. The pulp nozzle extrudes a third gas stream that contains an absorbent material, such as pulp fibers.

The first, second and third gas streams merge to form a fiber matrix. A nonwoven web is formed as a result of the meltblown, spunblown and pulp fibers collecting on the forming surface.

It was found that the nonwoven web formed by the aforementioned co-forming process has an increased cross-direction tensile strength, at least in part, because the spunblown fibers act as a reinforcing layer in the web.

In an additional embodiment, the nonwoven web includes a plurality of layers of meltblown and spunblown fibers. An absorbent material, such as pulp fibers, is dispersed in the layers of the thermoplastic polymer filaments. The spunblown fibers can be positioned between the meltblown fibers and can be mixed with less absorbent material than contained in the meltblown fiber layers. Three die head configurations or more can be used to form the nonwoven web in a single process. Alternatively, each layer of the coform nonwoven material can be formed in a separate step. The different layers can then be later combined together. In one aspect, each layer can be formed on top of a previously formed layer. The spunblown fiber layer positioned in the middle of the nonwoven web functions as a reinforcing layer while allowing the nonwoven web to still feel soft.

In one embodiment, the present disclosure is directed to a coform nonwoven. The coform nonwoven comprises a nonwoven fiber matrix comprising thermoplastic polymer fibers. The polymer fibers include meltblown fibers and spunblown fibers. The spunblown fibers have a greater fiber diameter than the meltblown fibers. The spunblown fibers comprise from about 10% to about 35% by weight of the thermoplastic polymer fibers. The meltblown fibers comprise from about 90% to about 65% by weight of the thermoplastic polymer fibers. The spunblown fibers form a layer within the fiber matrix. The coform nonwoven further includes a liquid absorbent material dispersed and attached to at least the meltblown fibers. The liquid absorbent material comprises from about 50% to about 90% by weight of the coform nonwoven.

In one aspect, the spunblown fibers and/or the meltblown fibers can comprise continuous filaments. The spunblown fibers can have a fiber diameter of from about 5 microns to about 50 microns, such as from about 10 microns to about 20 microns. The meltblown fibers can have a fiber diameter of less than about 10 microns, such as less than about 5 microns. The coform nonwoven can have a basis weight of from about 10 gsm to about 100 gsm, such as from about 20 gsm to about 80 gsm. The liquid absorbent material contained in the coform nonwoven can be pulp fibers, a superabsorbent material, or a mixture of both.

In one aspect, the liquid absorbent material forms a concentration gradient over the thickness of the coform nonwoven. For example, a minimum concentration of liquid absorbent material can be contained within the spunblown layer. A greater concentration of the liquid absorbent material can be contained in the meltblown layers.

In one aspect, the meltblown fibers can be formed from a first thermoplastic polymer while the spunblown fibers can be formed from a second thermoplastic polymer. The melt flow rate of the first polymer can be greater than the melt flow rate of the second polymer. For instance, the melt flow rate of the first polymer can be from about 30% to about 500% greater than the melt flow rate of the second polymer.

In one aspect, the coform nonwoven includes a first layer of meltblown fibers and a second layer of meltblown fibers, wherein the spunblown layer is positioned between the first layer of meltblown fibers and the second layer of meltblown fibers.

Coform nonwovens formed in accordance with the present disclosure generally have improved tensile strength in the cross-machine direction. In addition, the overall strength characteristics of the coform nonwoven are more uniform when comparing the strength in the machine-direction versus the strength in the cross-machine direction. For example, the coform nonwoven can have a machine direction/cross-machine direction tensile strength ratio of less than about 2.8, such as less than about 2.7 and greater than about 1, such as greater than about 1.5.

Other features and aspects of the present disclosure are discussed in greater detail below.

### BRIEF DESCRIPTION OF DRAWINGS

A full and enabling disclosure of the present disclosure is set forth more particularly in the remainder of the specification, including reference to the accompanying figures, in which:

FIG. 1 is a schematic illustrating one embodiment of one coforming process used in the present invention.

FIG. 2 is a cross-section view of one embodiment of a nonwoven web of the present invention.

FIG. 3 is a cross-section view of another embodiment of a nonwoven web of the present invention.

FIG. 4 is a cross-section view of an additional embodiment of a nonwoven web of the present invention.

Repeat use of reference characters in the present specification and drawings is intended to represent the same or analogous features or elements of the present invention.

### DEFINITIONS

When introducing elements of the present disclosure or the preferred embodiment(s) thereof, the articles “a”, “an”, and “the” when used herein are intended to mean that there are one or more of the elements.

The terms “comprising”, “including” and “having” when used herein are intended to be inclusive and mean that there may be additional elements other than the listed elements.

The term “absorbent article” when used herein includes, but is not limited to, personal care absorbent articles, such as baby wipes, mitt wipes, diapers, pant diapers, open diapers, training pants, absorbent underpants, incontinence articles, feminine hygiene products (e.g., sanitary napkins), swim wear and so forth; medical absorbent articles, such as garments, fenestration materials, underpads, bedpads, bandages, absorbent drapes, and medical wipes; food service wipers; clothing articles; pouches, and so forth. Materials and processes suitable for forming such articles are well known to those skilled in the art. An absorbent article, for example, can include a liner, an outer cover, and an absorbent material or pad positioned therebetween.

The term “basis weight” when used herein refers to the weight per unit surface area (in a machine-direction/cross-direction plane) of a sample of nonwoven web, expressed in grams/meter² (gsm). Basis weight may also be expressed in ounces of material per square yard (osy) and the fiber diameters are usually expressed in microns. (Note that to convert from osy to gsm, multiply osy by 33.91.)

The term “nonwoven web” when used herein means that the web comprises an intermixed and/or entangled blend of coformed material which comprises at least two different materials, such as meltblown fibers, spunblown fibers and cellulose or wood pulp fibers. A coformed nonwoven web may also comprise solid particulate additives such as but not limited to absorbent gel materials, filler particles, particulate spot bonding powders or clays.

The term “cross-machine direction” or “CD” when used herein with respect to a nonwoven web means the direction perpendicular to the predominant direction of movement of the nonwoven web structure through its manufacturing line (referred to as the “machine direction”) and can also be referred to as the width direction.

The term “fiber” when used herein means an elongate particulate having a length exceeding its width or diameter, e.g., a length to width ratio of greater than 10. A “fiber” can be continuous, such as a continuous filament, or discontinuous. One example of a discontinuous fiber is an elongate particulate that has a length of less than 3 cm. Non-limiting examples of discontinuous fibers include hardwood and softwood pulp fibers; hemp bast; bagasse; bamboo; corn stalk; cotton; cotton stalk; cotton linters; esparto grass; flax tow; jute bast; kenaf bast; reed; rice straw, sisal; switch grass; wheat straw; and synthetic staple (i.e., cut or chopped) fibers made from polyester, nylons, rayon (including viscose and lyocell), polyolefins such as polypropylene and polyethylene, natural polymers, such as starch, starch derivatives, cellulose and cellulose derivatives, hemicellulose, hemicellulose derivatives, chitin, chitosan, polyisoprene (cis and trans), peptides, polyhydroxyalkanoates, copolymers of polyolefins such as polyethylene-octene, and biodegradable or compostable thermoplastics such as polylactic acid, polyvinyl alcohol, and polycaprolactone. In addition to staple fibers, synthetic fibers can also be continuous. Synthetic fibers may be monocomponent or multicomponent, e.g., bicomponent.

The term “machine direction” or “MD” as used herein with respect to a nonwoven web means the direction parallel to the predominant direction of movement of the nonwoven web through its manufacturing line and can also refer to the length direction.

The term “MD/CD tensile ratio” when used herein refers to the machine direction tensile strength of a nonwoven web divided by the cross-machine direction tensile strength of the nonwoven web.

The term “melt flow rate” (MFR) when used herein is a measure of the ease of flow of the melt of a polymer composition. MFR is measured according to ISO 1133-1 and is described in the test method section below. MFR has units of g/10 minutes and is the measurement of the mass of a polymer, in grams, flowing in ten minutes through a capillary of a specific diameter and length by a pressure applied via prescribed alternative gravimetric weights for alternative prescribed temperatures.

The term “spunblown fiber” refers to macrofiber meltblown fibers, which are fibers formed by extruding a molten thermoplastic material through a plurality of fine, usually circular, die capillaries as molten threads or fibers into converging high velocity, usually hot, gas (e.g. air) streams which attenuate the filaments of molten thermoplastic material to reduce their diameter. The method is similar to forming conventional meltblown fibers, but differs in that the fibers have a larger diameter. More particularly, meltblown processes typically form microfibers (with average diameters less than about 5 microns), whereas spunblown processes typically form macrofibers (with average diameters from about 5 to about 50 microns). One example of a conventional meltblown method is disclosed, for example, in U.S. Pat. No. 3,849,241 to Butin et al., which is incorporated herein by reference in its entirety in a manner consistent with the present invention. As described above, spunblown fibers are macrofibers, which may be continuous or discontinuous, and are generally tacky when deposited onto a collecting surface.

The term “plurality” when used herein refers to two or more.

The term “tensile strength” when used herein refers to a measure of the ability of a material to withstand a longitudinal stress, expressed as the greatest stress that the material can stand without breaking. Tensile strength is expressed in grams per unit of force (gf).

The term “z-direction” wherein used herein means the direction orthogonal to the plane defined by the machine direction and cross-machine direction of the nonwoven web or fibrous web structure.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to coform nonwoven webs that contain different types of thermoplastic polymer fibers. For instance, the coform nonwoven web can contain at least one meltblown fiber layer and at least one spunblown fiber layer. An absorbent material, such as pulp fibers, is mixed with the thermoplastic polymer fibers. In one aspect, a greater concentration of the absorbent material is combined and attached to the meltblown fibers in comparison to the amount of absorbent material combined with the spunblown fibers. Conform nonwoven webs made according to the present disclosure have dramatically improved cross-directional strength. Although unknown, it is believed that the spunblown layer greatly enhances cross directional strength due to a combination of factors. For example, the spunblown fibers have a higher tenacity than the meltblown fibers. In addition, when less absorbent material is contained in the spunblown fiber layer, the spunblown fibers have a greater opportunity to bond together at intersection points for further improving cross directional strength. In addition, in some embodiments, it is believed that the spunblown fibers may have greater orientation in the cross-direction in comparison to the meltdown fibers, which can further improve cross-directional strength.

One aspect of the current invention relates to a method for manufacturing a nonwoven web that includes providing a forming surface that is traveling in the MD. Additionally, the present invention comprises one or more co-forming processes wherein each co-forming process includes a first and second meltblown die head disposed above the forming surface. The first meltblown die head extrudes a first gas stream and a second meltblown die head extrudes a second gas stream. The first gas stream includes meltblown fibers and the second gas stream includes spunblown fibers. Further, a pulp nozzle is disposed above and perpendicular to the forming surface. The pulp nozzle extrudes a third gas stream that contains pulp fibers and can be located between the first and the second gas streams.

It was found that the nonwoven web formed herein has an increased CD tensile strength. The increase in CD tensile strength in the nonwoven web may be attributed to the spunblown fibers. Tensile strength (procedure described below) was used herein to measure the CD peak load tensile strength. Accordingly, the nonwoven webs disclosed herein tend to exhibit greater CD strengths (the MD is the direction of movement, relative to the forming die, of the substrate on which the web is formed; the CD is perpendicular to the MD). Accordingly, the nonwoven web can have a CD Tensile Strength of at least about 50% greater compared to a substantially similar web prepared without using spunblown fibers in the co-forming process.

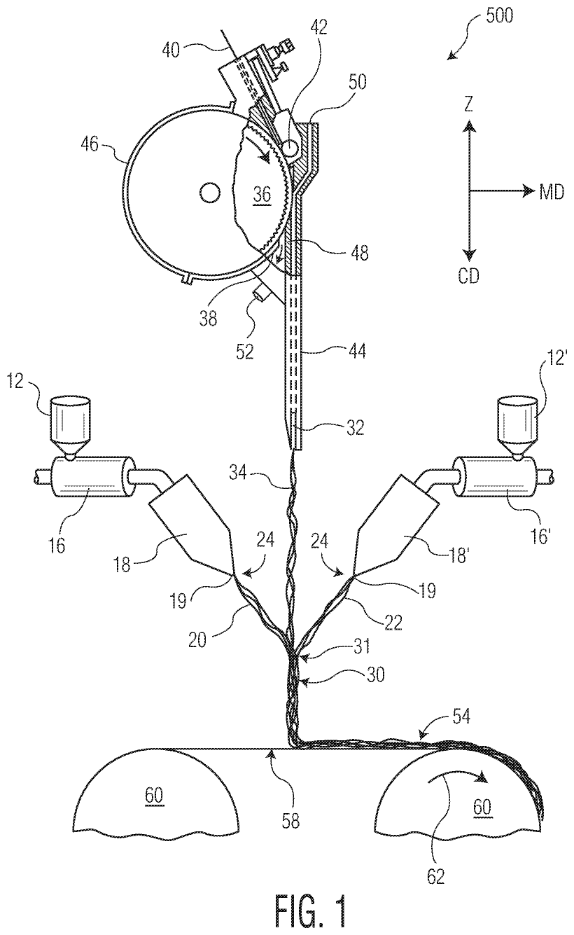

Referring to FIG. 1, one embodiment of a co-forming process 500 is shown for making a nonwoven web of the present invention. In this embodiment, the apparatus includes extruders 16 and 16′, respectively, into which a thermoplastic polymer composition may be introduced. The extruders 16 and 16′ each have an extrusion screw (not shown), which is driven by a conventional drive motor (not shown). As the polymer advances through the extruders 16 and 16′, it is progressively heated to a molten state due to rotation of the extrusion screw by the drive motor. Heating may be accomplished in a plurality of discrete steps with its temperature being gradually elevated as it advances through discrete heating zones of the extruders 16 and 16′ toward two meltblowing die heads 18 and 18′, respectively. The meltblowing die heads 18 and 18′ may constitute another heating zone where the temperature of the thermoplastic polymer is maintained at an elevated level for extrusion.

When two or more meltblowing die heads are used, such as described above, it should be understood that the fibers produced from the individual die heads may be different types of fibers. That is, the size, shape, or polymeric composition of the meltblown fibers yield smaller fibers in comparison with the spunblown fibers. Furthermore, the meltblown or spunblown fibers may be monocomponent and multicomponent. Accordingly, smaller fibers are produced by the first meltblowing die head 18. The first meltblowing die head 18 has an average diameter of about 5 micrometers or less, in some embodiments about 10 micrometers or less, and in some embodiments, from about 5 to about 50 micrometers or less in comparison with the second die head 18′.

In another aspect, it may be desirable to have the relative basis weight production skewed, such that first die head 18 is responsible for the majority of the basis weight of the thermoplastic polymer fiber portion of the nonwoven web being formed. As a specific example, about 70% by weight to about 85% by weight of the thermoplastic polymer fibers contained in the nonwoven web can be meltblown fibers produced from the first meltblowing die head 18, while the second meltblowing die head 18′ produces spunblown fibers that make up the remaining amount of thermoplastic polymer fibers contained in the coform nonwoven web material. Generally speaking, the overall basis weight of the nonwoven web, preferably coform, is from about 10 gsm to about 350 gsm. The basis weight of the coform nonwoven, for instance, can be greater than about 15 gsm, such as greater than about 20 gsm, such as greater than about 25 gsm, such as greater than about 30 gsm, and generally less than about 100 gsm, such as less than about 90 gsm, such as less than about 80 gsm, such as less than about 70 gsm, such as less than about 60 gsm, such as less than about 50 gsm.

Each meltblowing die head 18 and 18′ is configured so that two streams of attenuating gas per die converge to form a single stream of gas which entrains and attenuates molten threads 19 as they exit small holes or orifices 24 in each meltblowing die head, 18 and 18′. The molten threads 19 are formed into fibers or, depending upon the degree of attenuation, microfibers, of a small diameter which is usually less than the diameter of the orifices 24. Thus, each meltblowing die head 18 and 18′ has a corresponding single stream of a first gas 20 and a second gas 22, respectively. The gas streams 20 and 22 containing polymer fibers are aligned to converge at an impingement zone 31.

Referring again to FIG. 1, absorbent fibers 32 (e.g., pulp fibers) are added at the impingement zone 31 along with the first gas stream 20 and the second gas stream 22. Introduction of the absorbent fibers 32 into the two streams 20 and 22 of thermoplastic polymer fibers 30 is designed to produce a graduated distribution of absorbent fibers 32 within the combined gas streams 20 and 22 of thermoplastic polymer fibers 30. This may be accomplished by merging a third gas stream 34 containing the absorbent fibers 32 between the two gas streams 20 and 22 of thermoplastic polymer fibers 30 so that all three gas streams converge in a controlled manner. Because they remain relatively tacky and semi-molten after formation, the thermoplastic polymer fibers 30 may simultaneously adhere and entangle with the absorbent fibers 32 upon contact therewith to form a coherent nonwoven web 54.

As shown in FIG. 1, the meltblowing die heads 18 and 18′ can be arranged at a certain angle with respect to the forming surface 58, such as described in U.S. Pat. Nos. 5,508,102 and 5,350,624 to Georger et al. Each die head 18 and 18′, for instance, can be set at an angle ranging from about 30 to about 90 degrees, in some embodiments from about 35 degrees to about 80 degrees, and in some embodiments from about 45 degrees to about 65 degrees. The die heads 18 and 18′ may be oriented at the same or different angles. In fact, the texture of the nonwoven web 54 may actually be enhanced by orienting one die at an angle different than another die.

In one aspect, the die head 18 as shown in FIG. 1 can be at an angle of from about 35 degrees to about 55 degrees to the forming surface 58 so that the meltblown fibers and pulp fibers experience robust mixing. The die head 18′, on the other hand, can be at an angle of from about 70 degrees to about 90 degrees so that very little mixing occurs between the pulp fibers and the spunblown fibers. In this manner, the spunblown fibers are free to have greater fiber to fiber bonds, which can further enhance cross-directional strength.

To accomplish the merger of the pulp fibers with the thermoplastic polymer fibers, any conventional equipment may be employed, such as a picker roll 36 arrangement having a plurality of teeth 38 adapted to separate a mat or batt 40 of absorbent fibers into the individual absorbent fibers. When employed, the sheets or mats 40 of fibers 32 are fed to the picker roll 36 by a roller arrangement 42. After the teeth 38 of the picker roll 36 have separated the mat of fibers into separate absorbent fibers 32, the individual fibers 32 are conveyed toward the stream of thermoplastic polymer fibers through a pulp nozzle 44. A housing 46 encloses the picker roll 36 and provides a passageway or gap 48 between the housing 46 and the surface of the teeth 38 of the picker roll 36. A gas, for example, air, is supplied to the passageway or gap 48 between the surface of the picker roll 36 and the housing 46 by way of a gas duct 50. The gas duct 50 may enter the passageway or gap 48 at the junction 52 of the nozzle 44 and the gap 48. The gas is supplied in sufficient quantity to serve as a medium for conveying the absorbent fibers 32 through the pulp nozzle 44. The gas supplied from the duct 50 also serves as an aid in removing the absorbent fibers 32 from the teeth 38 of the picker roll 36. The gas may be supplied by any conventional arrangement such as, for example, an air blower (not shown). It is contemplated that additives and/or other materials may be added to or entrained in the gas stream 34 to treat the absorbent fibers 32. The individual absorbent fibers 32 are typically conveyed through the pulp nozzle 44 at about the velocity at which the absorbent fibers 32 leave the teeth 38 of the picker roll 36. In other words, the absorbent fibers 32, upon leaving the teeth 38 of the picker roll 36 and entering the nozzle 44, generally maintain their velocity in both magnitude and direction from the point where they left the teeth 38 of the picker roll 36. Such an arrangement, which is discussed in more detail in U.S. Pat. No. 4,100,324 to Anderson, et al.

As described above, in one embodiment, less pulp fibers can be incorporated into the spunblown fibers in relation to the amount of pulp fibers incorporated into the meltblown fibers. In addition to the die head angles, the velocity of the third gas stream 34 may also be adjusted in order to to place the pulp fibers in certain locations of the coform nonwoven web. For example, when the velocity of the third gas stream 34 is adjusted so that it is greater than the velocity of each stream 20 and 22 containing entrained thermoplastic polymer fibers 30 upon contact at the impingement zone 31, the absorbent fibers 32 are incorporated in the nonwoven web 54 in a gradient structure. That is, the absorbent fibers 32 have a higher concentration between the outer surfaces of the nonwoven web 54. For example, the minimum concentration of absorbent material or absorbent fibers 32 can be found in the spunblown layer contained within the coform nonwoven 54. On the other hand, when the velocity of the third gas stream 34 is less than the velocity of the first gas stream 20 and the second gas stream 22, the absorbent fibers 32 are incorporated in the nonwoven web 54 in a substantially homogenous fashion. That is, the concentration of the absorbent fibers 32 is substantially the same throughout the nonwoven web 54. This is because the low-speed stream of absorbent fibers 32 is drawn into a higher-speed stream of thermoplastic polymer fibers 30 to enhance turbulent mixing which results in a consistent distribution of the absorbent fibers 32.

To convert the composite stream of thermoplastic polymer fibers 30 and absorbent fibers 32 into a nonwoven web 54, a collecting device is located in the path of the composite stream. The collecting device may be a forming surface 58 (e.g., belt, drum, wire, fabric, etc.) driven by rollers 60 and that are rotating as indicated by the arrow 62 in FIG. 1. The merged streams of thermoplastic polymer fibers 30 and absorbent fibers 32 are collected as a fiber matrix on the surface of the forming surface 58 to form the nonwoven web 54. If desired, a vacuum box (not shown) may be employed to assist in drawing the near molten thermoplastic polymer fibers 30 onto the forming surface 58.

It should be understood that the present invention is by no means limited to the above-described embodiments. In an alternative embodiment, for example, first and second meltblowing die heads 18 and 18′ may be employed that extend substantially across a forming surface 54 in a direction that is substantially transverse to the direction of movement of the forming surface 54. As shown in FIG. 1, for instance, the machine direction 80 is parallel to the moving direction of the coform nonwoven 54, while the cross-machine direction 82 is perpendicular to the machine direction 80. The die heads 18 and 18′ may likewise be arranged in a substantially vertical disposition, i.e., perpendicular to the forming surface 54. so that the thus-produced fibers are blown directly down onto the forming surface 54. Such a configuration is well known in the art and described in more detail in, for instance, U.S. Patent Application Publication No. 2007/0049153 to Dunbar, et al.

In one aspect of the invention, any absorbent material such as absorbent fibers, particles, etc. may generally be employed through a pulp nozzle 44. The absorbent material includes fibers formed by a variety of pulping processes, such as kraft pulp, sulfite pulp, thermomechanical pulp, etc. The pulp fibers may include softwood fibers having an average fiber length of greater than 1 mm and particularly from about 2 to 5 mm based on a length-weighted average. Such softwood fibers may include, but are not limited to, northern softwood, southern softwood, redwood, red cedar, hemlock, pine (e.g., southern pines), spruce (e.g., black spruce), combinations thereof, and so forth. Exemplary commercially available pulp fibers suitable for the present invention include those available from Weyerhaeuser Co. of Federal Way, Wash. under the designation “Weyco CF-405” Hardwood fibers, such as eucalyptus, maple, birch, aspen, and so forth, can also be used. In certain instances, eucalyptus fibers may be particularly desired to increase the softness of the web. Eucalyptus fibers can also enhance the brightness, increase the opacity, and change the pore structure of the web to increase its wicking ability. Moreover, if desired, secondary fibers obtained from recycled materials may be used, such as fiber pulp from sources such as, for example, newsprint, reclaimed paperboard, and office waste. Further, other natural fibers can also be used in the present invention, such as abaca, sabai grass, milkweed floss, pineapple leaf, and so forth. In addition, in some instances, synthetic fibers may also be utilized.

Besides or in conjunction with pulp fibers, the absorbent material may also include a superabsorbent that is in the form fibers, particles, gels, etc. Generally speaking, superabsorbents are water-swellable materials capable of absorbing at least about 20 times its weight and, in some cases, at least about 30 times its weight in an aqueous solution containing 0.9 weight percent sodium chloride. The superabsorbent may be formed from natural, synthetic and modified natural polymers and materials. Examples used herein may include superabsorbent particles used as a cross-linked terpolymer of acrylic acid (AA), methylacrylate (MA) and a small quantity of an acrylate/methacrylate monomer. Alternatively, examples of synthetic superabsorbent polymers that may be used herein include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further, superabsorbents include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and so forth. Mixtures of natural and wholly or partially synthetic superabsorbent polymers may also be useful in the present invention. Particularly suitable superabsorbent polymers are HYSORB 8800AD (BASF of Charlotte, N.C. and FAVOR SXM 9300 (available from Degussa Superabsorber of Greensboro, N.C.).

In an additional aspect of the invention, the nonwoven web of the present invention can be made by a process in which at least one meltblown die head (e.g., two) is arranged near a chute through which the absorbent material is added while the web forms. Some examples of such techniques are disclosed in U.S. Pat. No. 4,100,324 to Anderson, et al., U.S. Pat. No. 5,350,624 to Georger, et al.; and U.S. Pat. No. 5,508,102 to Georger, et al., as well as U.S. Patent Application Publication Nos. 2003/0200991 to Keck, et al. and 2007/0049153 to Dunbar, et al., all of which are incorporated herein in their entirety by reference thereto for all purposes.

Additionally, it may be desired in certain cases to form a nonwoven web that is textured. Referring again to FIG. 1, for example, one embodiment of the present invention employs a forming surface 58 that is foraminous in nature so that the fibers may be drawn through the openings of the surface and form dimensional cloth-like tufts projecting from the surfaces of the material that correspond to the openings in the forming surface 58. The foraminous surface may be provided by any material that provides sufficient openings for penetration by some of the fibers, such as a highly permeable forming surface. Surface weave geometry and processing conditions may be used to alter the texture or tufts of the material. The particular choice will depend on the desired peak size, shape, depth, surface tuft “density” (that is, the number of peaks or tufts per unit area), etc. In one aspect, for example, the surface may have an open area of from about 35 percent to about 65 percent, in some embodiments from about 40 percent to about 60 percent, and in some embodiments, from about 45 percent to about 55 percent. One exemplary high open area forming surface is the forming surface FORMTECH™ 6 manufactured by Albany International Co. of Albany, N.Y. Such a surface has a “mesh count” of about six strands by six strands per square inch (about 2.4 by 2.4 strands per square centimeter), i.e., resulting in about 36 foramina or “holes” per square inch (about 5.6 per square centimeter), and therefore capable of forming about 36 tufts or peaks in the material per square inch (about 5.6 peaks per square centimeter). The FORMTECH™ 6 surface also has a warp diameter of about 1 millimeter polyester, a shute diameter of about 1.07 millimeters polyester, a nominal air permeability of approximately 41.8 m^{3/}min (1475 ft^{3/}min), a nominal caliper of about 0.2 centimeters (0.08 inch) and an open area of approximately 51 percent. Another exemplary forming surface available from the Albany International Co. is the forming surface FORMTECH™ 10, which has a mesh count of about 10 strands by 10 strands per square inch (about 4 by 4 strands per square centimeter), i.e., resulting in about 100 foramina or “holes” per square inch (about 15.5 per square centimeter), and therefore capable of forming about 100 tufts or peaks per square inch (about 15.5 peaks per square centimeter) in the material. Still another suitable forming surface is FORMTECH™ 8, which has an open area of 47 percent and is also available from Albany International. Of course, other forming wires and surfaces (e.g., drums, plates, etc.) may be employed. Also, surface variations may include, but are not limited to, alternate weave patterns, alternate strand dimensions, release coatings (e.g., silicones, fluorochemicals, etc.), static dissipation treatments, and the like. Still other suitable foraminous surfaces that may be employed are described in U.S. Patent Application Publication No. 2007/0049153 to Dunbar, et al.

The nonwoven web formed may be used in a wide variety of articles. For example, the nonwoven web may be incorporated into an “absorbent article” that is capable of absorbing water or other fluids.

Coform nonwovens made according to the present disclosure can include various different layers and constructions. For example, in one embodiment, the coform nonwoven can include a single spunblown layer combined with a single meltblown layer in which the absorbent material is primarily dispersed within the meltblown layer. In other embodiments, the coform nonwoven can include a plurality of meltblown layers combined with a single spunblown layer, a plurality of spunblown layers combined with a single meltblown layer, or a plurality of meltblown layers combined with a plurality of spunblown layers.

As shown in FIG. 1, coform non-woven webs made in accordance with the present disclosure can be formed in a single process step. Alternatively, different layers can be first formed and combined together in separate processes. For example, a meltblown coform layer can first be produced and then combined with a spunblown layer. The resulting non-woven web can then be combined with another coform meltblown layer such that the spunblown layer is positioned between the two coform meltblown layers. Separate layers can be formed on separate processes and then laminated together. Alternatively, one layer can be formed directly on top of another layer in forming the non-woven webs.

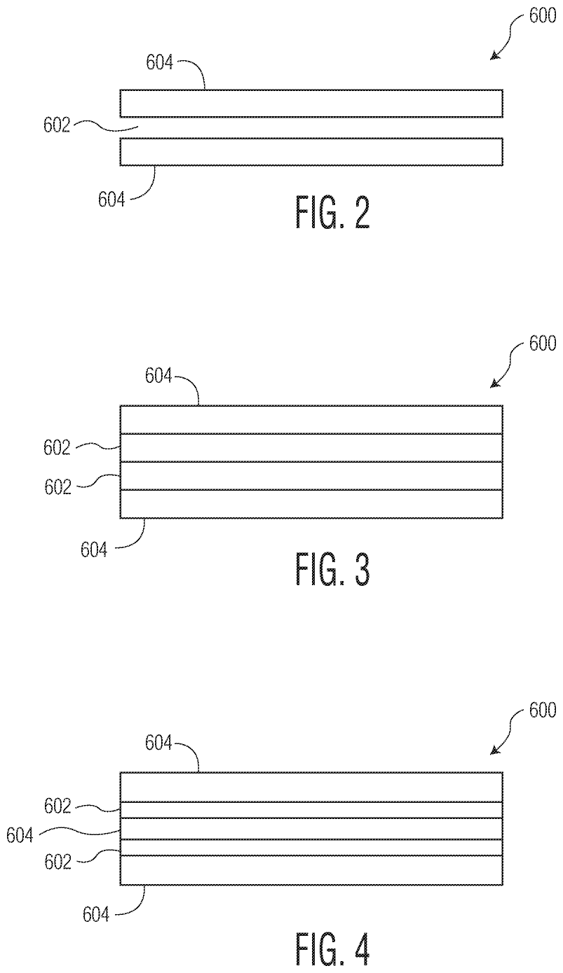

As shown in FIG. 2, in one aspect, a nonwoven web 600 includes a layer of spunblown fibers 602 and two layers of meltblown fibers 604, wherein the layers of meltblown fibers 604 are positioned outside of the layer of the spunblown fibers 602. Absorbent material, such as pulp fibers, can be distributed homogeneously throughout the web or can be present in greater concentrations in the meltblown fiber layers 604.

The spunblown layer 602 may be formed using conventional meltblowing technology. More particularly, the method of formation involves extruding a molten polymeric material into fine streams and attenuating the streams by opposing flows of high velocity, heated gas (usually air). Subsequent collection of the fibers on a foraminous screen belt, drum, or the like yields a layer of the spunblown fibers. The spunblown fiber layer 602 can be processed at a lower throughput than meltblown fiber layer 604. Operating at lower throughput allows formation of spunblown fibers at lower forming distances, such as about 3 to 4 inches. The spunblown layer possesses integrity due to entanglement of the individual fibers in the layer as well as some degree of thermal or self-bonding between the fibers, particularly when collection is effected only a short distance after extrusion. The resulting spunblown layer 602 is highly uniform and may be deposited onto a nonwoven web 600 at high manufacturing speeds. The fiber size may be controlled depending on the application. In liner applications, for example, the process conditions may be set to produce larger fiber sizes in order to enhance coverage without sacrificing intake properties. In general, the macrofibers contained in the spunblown layer 602 have an average fiber diameter between about 5 and about 50 microns, or between about 10 and about 20 microns and the spunblown fibers are predominantly continuous.

The meltblown layers 604 may be formed using conventional meltblowing technology, as known to those skilled in the art. While many different meltblown methods are known, these methods generally involve continuously extruding a thermoplastic polymer (either from the melt or a solution) through a spinneret in order to form discrete fibers. Thereafter, the fibers are drawn (either mechanically or pneumatically) without breaking in order to molecularly orient the polymer fibers and achieve tenacity. Lastly, the continuous fibers are deposited in a substantially random manner in the MD onto a carrier belt or the like to form a web of substantially continuous and randomly arranged, molecularly oriented fibers. The meltblown fibers generally have an average fiber diameter that is less than the average fiber diameter of the spunblown fibers. The meltblown fibers, for instance, generally have an average diameter of less than about 40 microns, such as less than about 30 microns, such as less than about 20 microns, such as less than about 15 microns, such as less than about 10 microns, such as less than about 8 microns, such as less than about 5 microns. The meltblown fibers generally have an average fiber diameter of greater than about 1 micron, such as greater than about 2 microns.

A wide variety of thermoplastic polymers may be used to prepare the spunblown layer 602 and the meltblown layers 604. The spunblown layer 602 and the meltblown layers 604 may be prepared from the same or different polymer types and two or more different polymers may be used in the preparation of either the spunblown layer 602 or the meltblown layers 604 or both. More particularly, the nonwoven fibers forming the spunblown layer 602 and the meltblown layers 604 may be monocomponent, bicomponent, or multi-component fibers. Thus, materials embodying the features of the invention may be fashioned with different physical properties by the appropriate selection of polymers or combinations thereof for the respective layers. Examples of suitable thermoplastic polymers include without limitation, polyolefins, polyamides, polyesters, polylactic acid (PLA) polycarbonates, polystyrenes, thermoplastic elastomers, fluoropolymers, vinyl polymers, and blends and copolymers thereof.

Suitable polyolefins include, but are not limited to, polyethylene, polypropylene, polybutylene, and the like; suitable polyamides include, but are not limited to, nylon 6, nylon 6/6, nylon 10, nylon 12 and the like; and suitable polyesters include, but are not limited to, polyethylene terephthalate, polybutylene terephthalate and the like. Particularly suitable thermoplastic polymers for use in the present invention are polyolefins including polyethylene, for example, linear low density polyethylene, low density polyethylene, medium density polyethylene, high density polyethylene and blends thereof; polypropylene; polybutylene and copolymers as well as blends thereof. Additionally, the suitable fiber forming polymers may have thermoplastic elastomers blended therein.

Although the spunblown fibers and the meltblown fibers can be made from the same thermoplastic polymer or from polymers having the same characteristics, as described above, in one embodiment, the polymer used to form the spunblown fibers can have a lower melt flow rate than the polymer used to produce the meltblown fibers. For example, the polymer used to produce the meltblown fibers can have a melt flow rate that is at least 30% greater, such as at least 50% greater, such as at least 70% greater, such as at least 100% greater, such as at least 150% greater, such as at least 200% greater, such as at least 250% greater, such as at least 300% greater, such as at least 350% greater, such as at least 400% greater than the melt flow rate of the polymer used to produce the spunblown fibers. The difference in melt flow rate can depend upon various factors, including the type of polymer used. When using polylactide polymers, for instance, the melt flow rate of the polymer used to produce the meltblown fibers can be twice as much or three times as much as the melt flow rate of the polymer used to produce the spunblown fibers. When using polyolefin polymers, such as polypropylene polymers, on the other hand, the polymer used to produce the meltblown fibers can be at least about 20% greater, such as at least about 30% greater, such as at least about 40% greater than the polymer used to produce the spunblown fibers.

The nonwoven web 600 may be composed substantially of the meltblown layers 604, and may include just a thin spunblown layer 602. For example, the nonwoven web 600 may have an overall basis weight between about 10 and about 100, or between about 12 and about 25, or between about 16 and about 21 grams per square meter (gsm). The nonowoven web 600 may include between about 2 percent and about 12 percent, or between about 4 percent and about 6 percent by weight spunblown material. As shown in FIG. 2, the layer of spunblown fiber 602 may be positioned between two layers of meltblown fiber 604. Other alternative embodiments include two layers of spunblown fibers 602 positioned in between two layers of meltblown fibers 604, as shown in FIG. 3. Or alternatively, two layers of spunblown fibers 602 positioned in an alternating fashion between three layers of meltblown fibers 604, as shown in FIG. 4. Any suitable combination of meltblown fiber layers 604 and spunblown fiber layers 602 may be included in the nonwoven web 600 so long as the layer of spunblown fibers 602 are positioned inside the meltblown layer of fibers 604.

### Test Methods

### Melt Flow Rate:

ISO standard 1133-1 test method cover the determination of the rate of extrusion of molten polymer resins using an extrusion plastometer. Generally, after a specified preheating time, resin is extruded through a die with a specified length and orifice diameter under prescribed conditions of temperature, load, and piston position in a coextruder. For simplicity, test method ISO standard 1131-1 is only described specifically herein since it is the method used in the disclosure.

ISO standard 1133-1: procedure for measurement of the melt flow rate (MFR). The procedure for determining MFR is as follows:
1. A small amount of the polymer sample (around 4 to 5 grams) is taken in a specially designed MFR apparatus. A die with an opening of typically around 2 mm diameter is inserted into the apparatus.
2. The material is packed properly inside the barrel to avoid formation of air pockets.
3. A piston is introduced which acts as the medium that causes extrusion of the molten polymer.
4. The sample is preheated for a specified amount of time: 5 min at 190° C. for a polyethylene and 6 min at 230° C. for a polypropylene.
5. After the preheating a specified weight is introduced onto the piston. Examples of standard weights are 2.16 kg, 5 kg, etc.
6. The weight exerts a force on the molten polymer and it immediately starts flowing through the die.
7. A sample of the melt is taken after the desired period of time and is weighed accurately.
8. MFR is expressed in grams of polymer per 10 minutes of duration of the test.

### Tensile Strength:

Testing of substrate should be conducted under TAPPI conditions (50 percent relative humidity, 73.degree. F.) with a procedure similar to ASTM-1117-80, section 7. Testing is conducted on a tensile testing machine maintaining a constant rate of elongation, and the width of each specimen tested was 3 inches. The “jaw span” or the distance between the jaws, sometimes referred to as gauge length, is 2.0 inches (76 mm). The crosshead speed is 12 inches per minute (304.8 mm/min.). A load cell or full-scale load is chosen so that all peak load results fall between 5 and 95 percent of the full-scale load. The break sensitivity is 70% and the slope preset points are 70 and 157 grams. Such testing may be done on an Instron 1122 tensile frame using MTS TESTWORKS for WINDOWS and Instron BLUEHILL software. This data system records at least 20 load and elongation points per second. Peak load (for tensile strength), peak energy and elongation at peak load (for stretch) are measured. At least ten samples for each test condition are tested and the average peak load or average stretch value is reported. For cross direction (CD) tensile tests, the sample is cut in the cross machine direction. For machine direction (MD) tensile tests, the sample is cut in the machine direction. The test method is used to test for peak load stretch on 25.4 mm wide and 152.4 mm long strips of wet or dry material.

### EXAMPLES

The following examples demonstrate some of the benefits and advantages of the present disclosure.

### Example No. 1

Various coform nonwovens were produced. More particularly, a coform nonwoven web was produced exclusively from meltblown fibers and compared to coform nonwovens made in accordance with the present disclosure in which a spunblown layer was positioned between two meltblown layers. The spunblown layer and coform meltblown layers were made in separate processes and combined together.

In this example, the meltblown fibers and spunblown fibers were made from a polylactide (PLA) polymer. The meltblown fibers were made from a combination of two polylactide polymers each having a melt flow rate of from 70 g/10 min to 85 g/10 min when tested at 210° C. (Poly 1 and Poly 2 in the Table below). The spunblown layers, on the other hand, were made from a polylactide polymer having a melt flow rate of 24 g/10 min when measured at 210° C.

The absorbent material used was pulp fibers. The following samples were produced:

| | | | | | | | | | Pulp | Primary | | |
|---|---|---|---|---|---|---|---|---|---|---|---|---|
| | Target | | | | | | | Melt | Nozzle | Air | Die | |
| Sample | B.W., | | B.W., | % | % | | | Temp. | Height, | Pressure, | Tip | |
| No. | gsm | Pulp/Poly | gsm | Polymer | Pulp | Poly-1 | Poly-2 | F. | inch | psi | angle, | PIH |
| 1 | 80 | 60/40 | 80 | 40 | 60 | 85 | 15 | 500 | 9.5 | 6 | 45 | 1.5 |
| 2 | 80 | 60/40 | 32.5 | 26 | 74 | 85 | 15 | 500 | 9.5 | 6 | 45 | 1.5 |
| | | | 15 | PLA Spunblown | | | | | | | | |
| | | | 32.5 | 26 | 74 | 85 | 15 | 500 | 9.5 | 6 | 45 | 1.5 |
| 3 | 70 | 65/35 | 27.5 | 17.2 | 82.8 | 85 | 15 | 500 | 9.5 | 6 | 45 | 1.5 |

| 15 | PLA Spunblown | | | | | | | | |
|---|---|---|---|---|---|---|---|---|---|
| 27.5 | 17.2 | 82.8 | 85 | 15 | 500 | 9.5 | 6 | 45 | 1.5 |

The above coform nonwovens were tested for tensile strength in the machine-direction and in the cross-machine direction. The following results were obtained:

| Sample No. | CD Peak Load (gf) | MD Peak Load (gf) |
|---|---|---|
| 1 | 345 | 1100 |
| 2 | 445 | 905 |
| 3 | 530 | 1190 |

As shown above, the nonwoven coforms made in accordance with the present disclosure had dramatically and unexpectedly better strength properties in the cross-machine direction. The coform nonwovens made in accordance with the present disclosure also had a better balance of strength properties. For instance, the samples made according to the present disclosure had a machine direction/cross-machine direction tensile ratio of from about 2 to about 2.3, whereas Sample No. 1 had a machine direction/cross-machine direction tensile ratio of greater than 3.

### Example No. 2

Further coform nonwovens were produced as described in Example No. 1 using polypropylene polymers. The following polypropylene polymers were incorporated into the samples:
Polymer 1—melt flow rate of 925 g/10 min
Polymer 2—melt flow rate of 1200 g/10 min
Polymer 3—melt flow rate of 500 g/10 min The samples were produced with pulp fibers. Each sample contained approximately 65% by weight pulp fibers and 35% by weight polymer fibers.

Two samples were produced containing only meltblown fibers. The remaining samples contained a spunblown layer positioned between two meltblown layers. The following samples were produced:

| | | | | | | | | | | | | | | Die |
|---|---|---|---|---|---|---|---|---|---|---|---|---|---|---|
| | | | | | | | | | Primary | Primary | | Tip | Pulp | Tip |
| | Basis | Basis | | | | MB | | Extruder | Air | Air | Die | to | Nozzle | to |
| Sample | wt., | wt., | | % | % | Bwt., | PIH: | Melt | Temp. | Pressure, | Tip | Tip, | Height, | Wire |
| No. | gsm | gsm | Polymer | Pulp | Polymer | gsm | MB | Temp | ° F. | psi | angle | inch | inch | inch |
| 4 | 70 | 70.0 | 1 | 65.0% | 35.0% | 24.5 | 1.5 | 410 | 410 | 5 | 45 | 6 | 9.5 | 7.5 |
| 5 | 70 | 70.0 | 2 | 65.0% | 35.0% | 24.5 | 1.5 | 390 | 390 | 6 | 45 | 6 | 9.5 | 7.5 |
| 6 | 70 | 30.0 | 1 | 76.0% | 24.0% | 7.2 | 1.5 | 410 | 410 | 6 | 45 | 6 | 9.5 | 7.5 |
| | | 10.0 | 1 | | 100.0% | 10.0 | 1.5 | 390 | 390 | 6 | 90 | 14 | — | 4 |
| | | 30.0 | 1 | 76.0% | 24.0% | 7.2 | 1.5 | 410 | 410 | 6 | 45 | 6 | 9.5 | 7.5 |
| 7 | 70 | 30.0 | 2 | 76.0% | 24.0% | 7.2 | 1.5 | 390 | 390 | 6 | 45 | 6 | 9.5 | 7.5 |
| | | 10.0 | 1 | | 100.0% | 10.0 | 1.5 | 390 | 390 | 6 | 90 | 14 | — | 4 |
| | | 30.0 | 2 | 76.0% | 24.0% | 7.2 | 1.5 | 390 | 390 | 6 | 45 | 6 | 9.5 | 7.5 |
| 8 | 70 | 30.0 | 2 | 76.0% | 24.0% | 7.2 | 1.5 | 390 | 390 | 6 | 45 | 6 | 9.5 | 7.5 |
| | | 10.0 | 3 | | 100.0% | 10.0 | 1.5 | 420 | 420 | 6 | 90 | 14 | — | 4 |
| | | 30.0 | 2 | 76.0% | 24.0% | 7.2 | 1.5 | 390 | 390 | 6 | 45 | 6 | 9.5 | 7.5 |
| 9 | 70 | 30.0 | 2 | 76.0% | 24.0% | 7.2 | 1.5 | 390 | 390 | 6 | 45 | 6 | 9.5 | 7.5 |
| | | 10.0 | 2 | | 100.0% | 10.0 | 1.5 | 390 | 390 | 6 | 90 | 14 | — | 4 |
| | | 30.0 | 2 | 76.0% | 24.0% | 7.2 | 1.5 | 390 | 390 | 6 | 45 | 6 | 9.5 | 7.5 |

The above coform nonwovens were tested for various physical properties. The following results were obtained:

| | | MD | MD | MD Total | | CD | CD | |
|---|---|---|---|---|---|---|---|---|
| | MD Peak | % Strain at | Energy To | Energy | CD Peak | % Strain | Energy | CD Total Energy |
| Sample | Load | Peak | Peak | Absorbed (TEA) | Load | at Peak | To Peak | Absorbed (TEA) |
| No. | gf | % | g*cm | gf*cm/cm\hat{ }2 | gf | % | g*cm | gf*cm/cm\hat{ }2 |
| 4 | 1400.0 | 19.5 | 1450.5 | 104.5 | 429.3 | 75.3 | 1729.4 | 109.7 |
| 5 | 1389.5 | 18.6 | 1277.3 | 89.2 | 419.3 | 70.9 | 1573.9 | 93.0 |
| 6 | 1192.3 | 24.1 | 1536.1 | 105.0 | 458.7 | 66.0 | 1637.1 | 93.3 |
| 7 | 1176.7 | 17.8 | 1184.1 | 77.1 | 456.4 | 64.6 | 1585.5 | 89.1 |
| 8 | 1121.5 | 19.6 | 1169.8 | 83.7 | 441.3 | 65.4 | 1550.7 | 90.5 |
| 9 | 1153.5 | 18.7 | 1190.0 | 82.7 | 446.4 | 49.2 | 1189.6 | 71.3 |

As shown above, adding a spunblown layer increases strength in the cross-machine direction. In addition, the samples made according to the present disclosure had better uniform properties in both the machine direction and the cross-machine direction. For example, for the samples made according to the present disclosure, the machine direction/cross-machine direction tensile strength ratio was from about 2.4 to about 2.7. The samples made exclusively with meltblown fibers, however, had a machine direction/cross-machine direction tensile strength ratio of much greater than 3.

### Example No. 3

Further coform nonwoven samples were produced as described in Example No. 1. In this example, all of the polymer fibers were formed from a polypropylene polymer having a melt flow rate of 925 g/10 min. The absorbent material was fluff pulp fibers. Each sample was formulated so as to contain 65% by weight pulp fibers overall and 35% by weight polymer fibers.

The following samples were produced:

| | | | | | | | | | | | | | Die |
|---|---|---|---|---|---|---|---|---|---|---|---|---|---|
| | | | | | | | | Primary | Primary | | Tip | Pulp | Tip |
| | Basis | Basis | | | MB | | Extruder | Air | Air | Die | to | Nozzle | to |
| Sample | Wt., | wt., | % | % | Bwt., | PIH: | Melt | Temp. | Pressure, | Tip | Tip, | Height, | Wire, |
| No. | gsm | gsm | Pulp | Polymer | gsm | MB | Temp | ° F. | psi | angle | inch | inch | inch |
| 10 | 70.0 | 70.0 | 65% | 35% | 24.5 | 1.5 | 410 | 410 | 6 | 45 | 6 | 9.5 | 7.5 |
| 11 | 70 | 30.0 | 76% | 24% | 7.2 | 1.5 | 410 | 410 | 6 | 45 | 6 | 9.5 | 7.5 |
| | | 10.0 | 0% | 100% | 10.0 | 1.5 | 390 | 390 | 6 | 90 | 14 | — | 4 |
| | | 30.0 | 76% | 24% | 7.2 | 1.5 | 410 | 410 | 6 | 45 | 6 | 9.5 | 7.5 |
| 12 | 70 | 30.0 | 76% | 24% | 7.2 | 1.5 | 410 | 410 | 6 | 45 | 6 | 9.5 | 7.5 |
| | | 10.0 | 0% | 100% | 10.0 | 1.5 | 370 | 370 | 6 | 90 | 14 | — | 4 |
| | | 30.0 | 76% | 24% | 7.2 | 1.5 | 410 | 410 | 6 | 45 | 6 | 9.5 | 7.5 |
| 13 | 70 | 30.0 | 76% | 24% | 7.2 | 1.5 | 410 | 410 | 6 | 45 | 6 | 9.5 | 7.5 |
| | | 10.0 | 0% | 100% | 10.0 | 1.5 | 410 | 410 | 6 | 45 | 6 | | 7.5 |
| | | 30.0 | 76% | 24% | 7.2 | 1.5 | 410 | 410 | 6 | 45 | 6 | 9.5 | 7.5 |

The above samples were tested for various physical properties and the following results were obtained:

| | MD | | | | CD | | | |
|---|---|---|---|---|---|---|---|---|
| | | | | Total | | | | Total |
| | | | | Energy | | | | Energy |
| | Peak | % Strain | Energy | Absorbed | Peak | % Strain | Energy | Absorbed |
| Sample | Load | at Peak | To Peak | (TEA) | Load | at Peak | To Peak | (TEA) |
| No. | gf | % | g*cm | gf*cm/cm\hat{ }2 | gf | % | g*cm | gf*cm/cm\hat{ }2 |
| 10 | 1340.0 | 21.8 | 1621.6 | 121.8 | 400.7 | 90.1 | 1910.8 | 114.3 |
| 11 | 1205.2 | 27.3 | 1947.0 | 128.8 | 460.0 | 75.2 | 1932.6 | 111.8 |
| 12 | 1121.5 | 28.5 | 1878.7 | 122.4 | 447.9 | 84.6 | 2077.9 | 117.8 |
| 13 | 1397.3 | 29.3 | 2367.4 | 160.9 | 430.0 | 97.8 | 2257.5 | 137.6 |

Consistent with Example Nos. 1 and 2 above, the inclusion of a spunblown layer greatly and dramatically enhances the cross-machine directional strength of the coform nonwoven.

First Embodiment: In a first embodiment the invention provides for a method for manufacturing a nonwoven web, the method comprises providing a forming surface traveling in a machine direction and the method comprises a co-forming process wherein:
a. providing a first and second die head disposed above the forming surface;
b. extruding a first gas stream comprising meltblown fibers from the first die head;
c. extruding a second gas stream comprising spunblown fibers from the second die head;
d. providing a pulp nozzle disposed above and perpendicular to the forming surface;
e. providing a third gas stream through the pulp nozzles positioned between the first and the second gas streams;
f. merging the first, second and third gas streams into a fiber matrix;
g. collecting the first gas stream fibers and collecting the second gas stream on the forming surface to form the nonwoven web.

The method according to the preceding embodiment, wherein the first and second die heads are disposed at an angle to the forming surface, wherein the first die head is directed more towards the third gas stream.

The method according to the preceding embodiments, wherein extruding a first gas stream from the first die head further comprises pulp fibers.

The method according to the preceding embodiments, wherein the nonwoven web has increased cross-machine direction tensile strength of greater than about 50% in comparison to a web not containing the spunblown fibers.

The method according to the preceding embodiments, wherein an amount of spunblown fibers is from 3% to about 20% of the web.

The method according to the preceding embodiments, wherein the nonwoven web is used in an absorbent article.

Second Embodiment: In a second embodiment the invention provides for a nonwoven with a plurality of fibers, wherein the nonwoven web has MD/CD Tensile Ratio less than about 2.8.

The nonwoven web according to the preceding embodiment, wherein the nonwoven web has a machine-direction/cross-machine direction tensile ratio ranging from about 1 to about 2.7.

The nonwoven web according to the preceding embodiments, wherein the nonwoven web is used in an absorbent article.

Third Embodiment: In a third embodiment, the invention provides for a coform nonwoven web comprising: a plurality of layers of meltblown fibers; a plurality of layers of spunblown fibers wherein the layers of spunblown fibers are positioned between the layers of the meltblown fibers.

The nonwoven web according to the preceding embodiments, wherein the layer of spunblown fibers comprises fibers having an average diameter between about 4 and about 30 microns.

The nonwoven web according to the preceding embodiments, wherein the layer of spunblown fibers comprise multicomponent fibers.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A coform nonwoven comprising: a nonwoven fiber matrix comprising thermoplastic polymer fibers, the thermoplastic polymer fibers including meltblown fibers and spunblown fibers, the spunblown fibers having a greater fiber diameter than the meltblown fibers, the spunblown fibers comprising from about 10% to about 35% by weight of the thermoplastic polymer fibers, the meltblown fibers comprising from about 90% to about 65% by weight of the thermoplastic polymer fibers, the spunblown fibers forming a layer within the fiber matrix; and a liquid absorbent material dispersed and attached to at least the meltblown fibers, the liquid absorbent material comprising from about 50% to about 90% by weight of the coform nonwoven.

2. The coform nonwoven as defined in claim 1, wherein the coform nonwoven has a machine-direction/cross-machine direction tensile ratio of less than about 2.8, such as less than about 2.7, and greater than about 1, such as greater than about 1.5.

3. coform nonwoven as defined in any of the preceding claims, wherein the spunblown fibers have a diameter of from about 5 microns to about 50 microns, such as from about 10 microns to about 20 microns.

4. The coform nonwoven as defined in claim 1, wherein the coform nonwoven has a basis weight of from about 10 gsm to about 100 gsm, such as from about 20 gsm to about 80 gsm.

5. The coform nonwoven as defined in claim 1, wherein the meltblown fibers comprise continuous filaments and the spunblown fibers comprise continuous filaments.

6. The coform nonwoven as defined in claim 1, wherein the spunblown fibers have a greater tenacity than the meltblown fibers.

7. The coform nonwoven as defined in claim 1, wherein the fiber matrix includes a first layer of meltblown fibers and a second layer of meltblown fibers and wherein the layer of spunblown fibers is positioned between the first layer of meltblown fibers and the second layer of meltblown fibers.

8. The coform nonwoven as defined in claim 1, wherein the absorbent material forms an absorbent material concentration gradient over a thickness of the coform nonwoven and wherein a minimum concentration of absorbent material is contained in the spunblown layer.

9. The coform nonwoven as defined in claim 1, wherein the liquid absorbent material comprises pulp fibers.

10. The coform nonwoven as defined in claim 1, wherein the liquid absorbent material comprises a superabsorbent material.

11. The coform nonwoven as defined in claim 1, wherein the meltblown fibers are formed from a first thermoplastic polymer having a melt flow rate and the spunblown fibers are formed from a second thermoplastic polymer having a melt flow rate and wherein the meltflow rate of the second thermoplastic polymer is less than the melt flow rate of the first thermoplastic polymer.

12. The coform nonwoven as defined in claim 11, wherein the melt flow rate of the first thermoplastic polymer is from about 30% to about 500% greater than the melt flow rate of the second thermoplastic polymer.

13. An absorbent article including an outer cover, an inner lining, and an absorbent pad positioned between the outer cover and the inner lining, and wherein the absorbent article incorporates the coform nonwoven as defined in claim 1.

14. A method for manufacturing a nonwoven web, wherein the method comprises providing a forming surface traveling in a machine direction and wherein the method comprises: a. providing a first and second die head disposed above the forming surface; b. extruding a first gas stream comprising meltblown fibers from the first die head; c. extruding a second gas stream comprising spunblown fibers from the second die head; d. providing a pulp nozzle disposed above and perpendicular to the forming surface; e. providing a third gas stream through the pulp nozzle positioned between the first and the second gas streams; and f. merging the first, second and third gas streams into a fiber matrix that forms a coform nonwoven web on the forming surface.

15. The method according to claim 14, wherein the first and second die heads are disposed at an angle to the forming surface.

16. The method according to claim 14, wherein extruding a first gas stream from the first die head further comprises pulp fibers.

17. The method according to claim 14, wherein the nonwoven web has a machine- direction/cross-machine direction tensile ratio of less than about 2.8, such as less than about 2.7, and greater than about 1, such as greater than about 1.5.

18. The method according to claim 14, wherein the amount of spunblown fibers based on a total weight of meltblown fibers and spunblown fibers present is from about 10% to about 35% by weight, the nonwoven web containing pulp fibers in an amount from about 50% to about 90% by weight.

19. The method according to claim 14, wherein the nonwoven web is used in an absorbent article.
